(19) United States
(12) Patent Application Publication
(10) Pub. No.: US 2020/0360608 A1
(43) Pub. Date: Nov. 19, 2020

(54) PUSH-PULL MEDICATION CONTAINER ADAPTER CAP FOR ENTERAL SYRINGE FILLING SYSTEMS

(71) Applicants: Nicholas J Perazzo, Rosedale, MD (US); Robert A Rosen, Owings Mills, MD (US)

(72) Inventors: Nicholas J Perazzo, Rosedale, MD (US); Robert A Rosen, Owings Mills, MD (US)

(21) Appl. No.: 16/893,737

(22) Filed: Jun. 5, 2020

Related U.S. Application Data
(62) Division of application No. 15/274,702, filed on Sep. 23, 2016, now abandoned.

(51) Int. Cl. A61M 5/178; A61M 39/24; A61M 39/20; B65B 7/28; B65B 5/04; B65B 3/28; B65B 3/30; B65B 3/00; B65B 57/02; G16H 20/17

(52) U.S. Cl. CPC A61M 5/1782; A61M 39/24; A61M 39/20; B65B 7/28; B65B 5/045; A61J 7/0053; B65B 3/30; B65B 3/003; B65B 57/02; G16H 20/17; A61M 2039/2486; B65B 3/28

(57) ABSTRACT

A medication container adapter that utilizes a push-pull valve for adapting medication containers for use with syringes that are filled on semi-automatic and automatic filling machinery. The syringes are filled when the medication container is in the inverted filling position. The push-pull valve comprises of a stationary portion and a poppet slidably inserted into the stationary portion. The poppet is an annular member having a central channel and a fluted axial outlet from the central channel. The push-pull valve can be configured to be pressed onto an existing medication container adapter or manufactured as an integrated part of the medication container adapter so that it can either be screwed onto or pressed into the medication container neck.

### CROSS-REFERENCE TO RELATED APPLICATIONS

The present application is a division of U.S. application Ser. No. 15/274,702 filed 2016 Sep. 23.

### BACKGROUND OF THE INVENTION

### 1. Field of the invention

The present invention relates generally to enteral syringe packaging equipment and more specifically to an adapter cap incorporating a push-pull valve optimized for use in a fully or partially automated system for preparing patient-specific doses of selected pharmaceutical liquid medication for administration by syringe.

### 2. Description of the Background

Oral syringes are used to dispense liquids into the mouth. Enteral syringes are used to dispense liquids into the gastro-intestinal tract through a tube. As shown in FIG. 1, oral syringes utilize a male tapered tip. Enteral syringes utilize a luer-lock tip as shown in FIGS. 2A & 2B. Both types of syringes are typically filled with medication on-demand at hospital pharmacies, from bulk medication containers supplied by OEM manufacturers. The hospitals and pharmacies maintain health records identifying the prescribed patient, prescribing physician, and prescription dosages and schedule. The OEM medication manufacturers also maintain important records for each bulk container including batch number, expiry date, storage instructions, product name, strength, name of the active ingredient(s), dose form, warning statements, National Drug Code (NDC), requirements to be shaken, refrigerated, protected from light, recalls, etc. However, hospital health records lack the OEM information and vice versa. Thus, hospital pharmacists seeking to fill oral and enteral syringes with medication on-demand at hospital pharmacies from bulk medication containers supplied by OEM manufacturers often lose considerable time contacting the OEM manufacturer for this information. The problem becomes even more apparent as the filling process becomes more automated. What is needed is a smart-container for bulk medicine that carries the OEM information with it.

Hospital pharmacists prefer to fill just one type of syringe because they typically do not know whether a prescription will ultimately be administered orally or enterally. Another issue of great concern relates to mistaken use of syringes. Syringes intended to be dispensed into the mouth or into an enteral tube can, inadvertently, though rarely, be dispensed into an intravenous (IV) patient port. Such mistakes have caused injury and death to patients. Both of these problems have recently been addressed by the Global Device Suppliers Association (GEDSA). This industry group has introduced new devices referred to as ENFIT™ which include enteral syringes. The ENFIT™ devices are not compatible with luer connections or any other type of small bore medical connectors. This ENFIT™ design prevents the misconnection of enteral syringes to the patient's tubing port. As a result of the GED5A organizations efforts, the International Standards Organization created ISO CD 803069-3 which specifies the safe design for an enteral feeding connection. The new ENFIT™ enteral syringes comply with ISO CD 80369-3, can be used to administer medication orally or enterally, and cannot easily be connected to an incorrect patient tubing port.

As shown in FIG. 1, oral syringes have a tapered tip which can be used with a medication container to syringe adapter with an elastomer valve for filling the syringe when the container is in the inverted position.

The tapered tip of the oral syringe (See FIG. 1) penetrates the elastomer valve which enables the syringe to extract the liquid from the medication container. This adapter arrangement is not suitable for filling enteral syringes as the enteral syringe has a female fitting on the tip and is not compatible with the elastomer valve adapter used for the oral syringe.

To address this problem, a new Push-Pull Valve Adapter (for which this patent application is based) was developed by the Inventors specifically for use with the female luer tip of the enteral syringe (refer to FIGS. 5 & 6).

Prior to filling the syringe, the valve is closed (FIG. 5). During the filling of the syringe, a mechanical actuator FIG. 5 (valve opener/closer), located at the fill station, opens the valve FIG. 6. When the syringe has been filled, the same mechanical actuator closes the valve.

The initial design of the enteral syringe tip caused excess liquid to accumulate in the tip of the syringe which caused unacceptable fill accuracy for the smaller size enteral syringes. A new design which incorporates an inner nozzle within the syringe discharge port addressed this problem (FIG. 5).

The change in the design of the discharge port of the enteral syringe required a new design for the syringe to medication adapter valve. This design changes obviated the prior art. A new adapter needed to fill both the smaller enteral syringes that had the center nozzle port (FIG. 5) as well as the larger syringes which did not have the center nozzle port (FIG. 11) without using change parts.

Thus, as shown in FIG. 2, two versions of enteral syringes now exist. As shown in FIG. 2(A) larger enteral syringes (typically 5 cc and above) utilize an internally-threaded female luer-lock tip and no center nozzle. As shown in FIG. 2(B) smaller enteral syringes (typically 0.5 ml-3 ml) utilize an internal tapered or slip-nozzle with an enteral threaded female luer-lock and a center nozzle.

Automated filling systems have been developed by Baxa, Inc., For Health Technologies, Inc., Intelligent Hospital Systems, Applicant National Instruments Co. (see, for example, Applicant's U.S. Ser. No. 13/788,849 filed Mar. 7, 2013 and others for the automated filling of syringes). However, the degree of automation in the hospital pharmacy for the packaging of oral/enteral syringes is limited due to the wide array of different syringes to fill, the different medicine containers to fill them from, and the lack of OEM information on bulk medication containers supplied by OEM manufacturers. Bulk medications are commonly provided in variously sized bottles or containers having threaded screw caps that must be removed and replaced with container-to-syringe adapter caps. Most any attempt at automated filling of any syringes requires a modification to standard manufacturer-supplied medicine containers. Semi-automated and automated filling of syringes requires that the medication container be in the inverted position while the syringe is being filled. It is also necessary that the medication container cap be open during the time that the syringe is being filled, and closed when the syringe has been filled to prevent leakage. Thus, the manufacturer-supplied screw-on caps must be removed and replaced by an adapter cap that allows the syringe to be connected to the medication container. To fill oral syringes it is known to use an adapter cap with an elastomeric valve that allow the syringe tip (FIG. 1) to penetrate the inverted container. The tapered tip of the oral syringe would penetrate the elastomer valve while the medicine container is held in an inverted position, enabling the syringe to extract the liquid from the medication container without leakage.

Unfortunately, the elastomeric seal-type adapter cap does not work with enteral syringes which are manufactured in a variety of sizes with differing plunger configurations (FIGS. 2A, 2B). The prior art adapter arrangement is not suitable for filling enteral syringes (FIGS. 2A, 2B) since the enteral syringe has a female fitting on the tip and is not compatible with the elastomer valve adapter used for the oral syringe.

Given the diversity of enteral syringes and medicines available, any semi-automated (or fully-automated) system will need sufficient dexterity to manipulate all the myriad prescription bottles containing the pharmaceuticals to be dispensed as well as variously sized enteral syringes, bringing them together in a controlled environment to quickly and accurately fill and label each syringe and to verify its work as it proceeds in order to avoid errors in the process. Existing adapter caps are incapable of use with enteral syringes that are being filled on an automated or semi-automated basis when the medication container is in the inverted position. Consequently, existing adapter caps do not address the needs of medical institutions desiring a semi-automatic or automatic enteral syringe filling system when the medication container is in the inverted position. In addition, an improved OEM bulk container is needed that includes batch number, expiry date, storage instructions, product name, strength, name of the active ingredient(s), dose form, warning statements, National Drug Code (NDC), requirements to be shaken, refrigerated, protected from light, recalls, etc., e.g., a smart-container that carries the foregoing OEM information with it. The present invention was developed to fill these voids.

### SUMMARY OF THE INVENTION

To address the need to fill enteral syringes, on a semi-automatic or automated basis, with the medicine container held in the inverted filling position, a push-pull adapter cap is herein disclosed. When filling the syringe the valve is open. After the syringe has been filled the valve is closed. The push-pull valve is open or closed by a mechanical actuator at the automated or semi-automated filling station.

The push-pull adapter cap disclosed herein, when used with an enteral syringe filling system, enables hospital pharmacists to simplify and streamline their task, increasing the number of prescriptions that can be filled in a day, and improving patient safety and care by minimizing medication errors and the consequences that ensue.

In addition, a smart-container for bulk medicine that carries OEM information with it is provided, including batch number, expiry date, storage instructions, product name, strength, name of the active ingredient(s), dose form, warning statements, National Drug Code (NDC), requirements to be shaken, refrigerated, protected from light, recalls, etc. This way, hospital pharmacists seeking to fill oral and enteral syringes with medication on-demand at hospital pharmacies from bulk medication containers supplied by OEM manufacturers have the OEM information at their disposal when filling the syringes, saving considerable time, effort and cost.

### BRIEF DESCRIPTION OF THE DRAWINGS

The objects, features, and advantages of the present invention will become apparent from the following detailed description of the preferred embodiments and certain modifications thereof when taken together with the accompanying drawings in which like numbers represent like items throughout and in which:

FIG. 1 is a perspective view of a conventional oral syringes with tapered tip.

FIG. 2(A) is a perspective view of a large-sized conventional enteral syringe with internally-threaded luer-lock tip.

FIG. 2(B) is a perspective view of a smaller-sized conventional enteral syringe with an internal tapered or slip-nozzle and an enteral threaded female luer-lock.

FIG. 3 is a perspective exploded view of a push-pull adapter cap 10 for retrofit application to a standard manufacturer-supplied medication container cap such as a Baxa™ or equivalent valve-less medicine container adapter cap with opening.

FIG. 4 is a perspective view of the push-pull adapter cap 10 of FIG. 3 illustrating how it interfaces an automated filling system.

FIGS. 5-6 are sequential perspective views of the push-pull adapter cap 10 of FIGS. 3-4 illustrating how it interfaces a smaller-sized enteral syringe (with internal tapered slip-nozzle and enteral threaded female luer-lock) while in the automated filling system.

FIG. 7 is a side view of the poppet 84 used in the push-pull adapter cap 10 of FIGS. 3-6.

FIG. 8 is an end cross-section of the poppet valve of FIG. 7.

FIG. 9 is a side cross-section of the press-on ring 60 used for attaching the push-pull adapter cap 10 of FIGS. 3-8 to a standard manufacturer-supplied medication container adapter cap.

FIG. 10 is a side cross-section of a modified press-in version used for attaching the push-pull adapter cap 10 inside the neck of a standard manufacturer-supplied medication container cap.

FIGS. 11-12 are sequential perspective views of the push-pull adapter cap 10 of FIGS. 3-6 illustrating how it interfaces a larger-size enteral syringe lacking a center nozzle, thereby illustrating the device's ability to adapt to both small syringes (with nozzle) and large syringes (w/o center nozzle).

FIG. 13 is a cross-section of the poppet valve of the present invention inserted into a large enteral syringe and illustrating the poppet valve circumferential seal surfaces on the large enteral syringe in accordance with the invention. FIG. 14 is a cross-section of the poppet valve of the present invention inserted into a small enteral syringe and illustrating the poppet valve circumferential seal surfaces on the small enteral syringe in accordance with the invention.

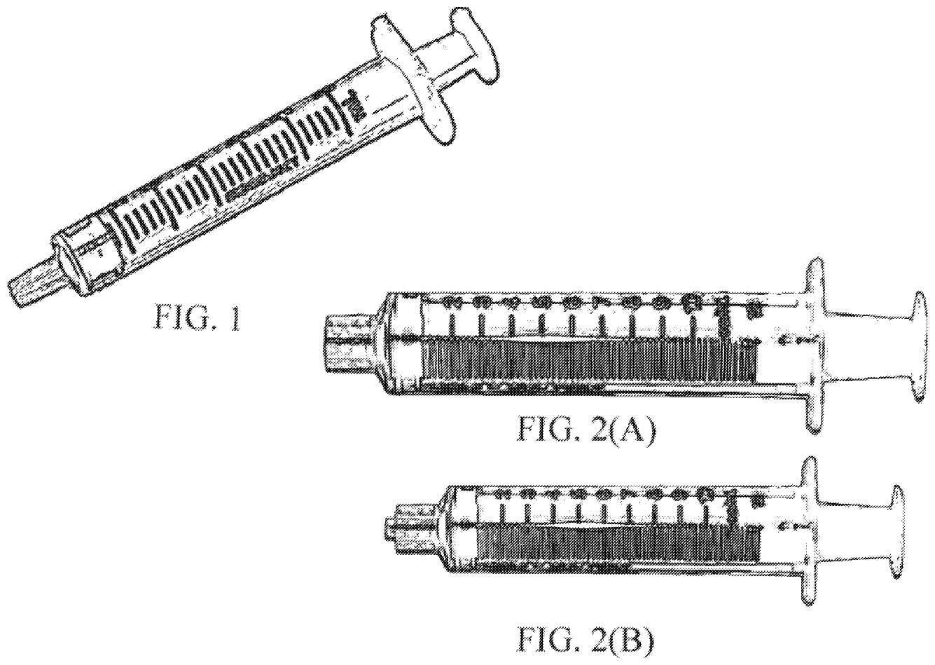

### DETAILED DESCRIPTION OF THE PREFERRED EMBODIMENTS

For the purposes of promoting an understanding of the principles of the invention, reference will now be made to the exemplary embodiment illustrated in the drawings and described below. The embodiment disclosed is not intended to be exhaustive or limit the invention to the precise form disclosed in the following detailed description. Rather, the embodiment is chosen and described so that others skilled in the art may utilize its teachings. It will be understood that no limitation of the scope of the invention is thereby intended. The invention includes any alterations and modifications in the illustrated device, the methods of operation, and further applications of the principles of the invention which would normally occur to one skilled in the art to which the invention relates.

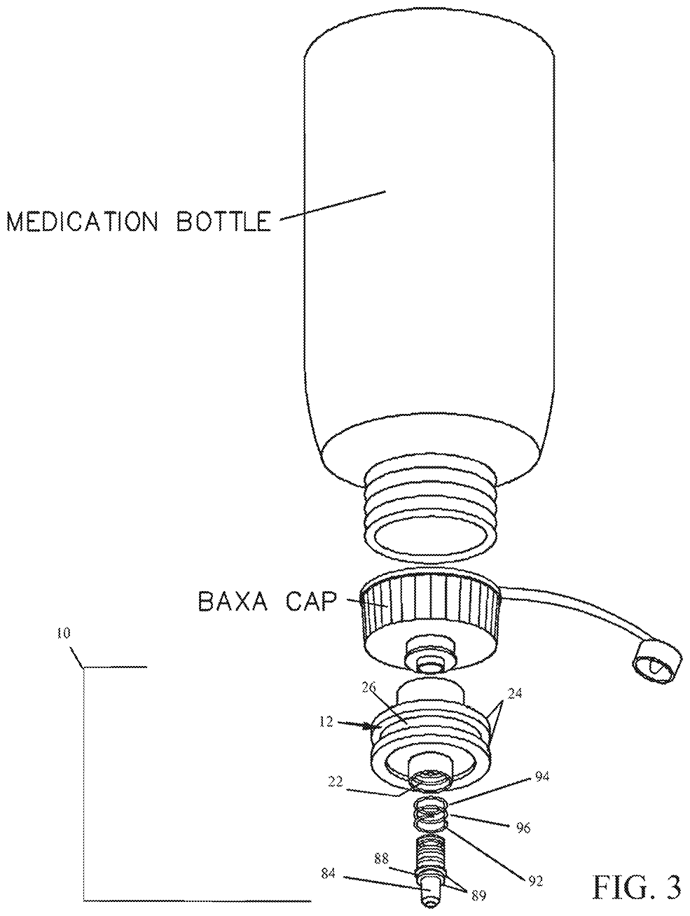

FIG. 3 is a perspective exploded view of a push-pull adapter cap 10 for retrofit application to a standard manufacturer-supplied (Baxa™ or Baxa equivalent) medicine container cap or equivalent valve-less medicine container cap with opening. For purposes of definition, this pre-existing medicine container cap is herein designated the “OEM cap.” Adapter cap 10 generally comprises a stationary portion 12 for attachment to the OEM cap, a poppet 84 slidably inserted into the stationary portion 12, a plurality of 0-rings inclusive of a fluid seal 94 and two poppet seals 92, 96.

Stationary portion 12 comprises an annular body with a central passage 22 there through, the central passage 22 being defined by an inner wall having a specific progression of diametric variations. At one end of the body of stationary portion 12, where stationary portion 12 attaches to the top of the manufacturer-supplied medicine container cap, central passage 22 has a relatively large diameter sized to conform to and receive the nozzle of the manufacturer-supplied medicine container cap as described below with reference to FIG. 8. This forms a “press-fit” seal. It will be understood by one of ordinary skill in the art, however, that stationary portion 12 may be sized to fit other manufacturer-supplied caps by different means based on design preference, such as a screw-on connection.

Central passage 22 continues partially through stationary portion 12 at a relatively constant diameter but then constricts at a shoulder 98 to a smaller diameter sized to accommodate the elastomer poppet 84, as will be described further herein, which protrudes from the distal end of passage 22. The outer wall of stationary portion 12 further comprise two spaced annular flanges 24 separated by an annular groove 26 for alignment with a stationary yoke of the filling station, as further described herein. Similarly, the outer wall of poppet 84 is equipped with two spaced annular flanges 89 separated by an annular groove 88 for alignment with a movable yoke of the filling station, as further described herein.

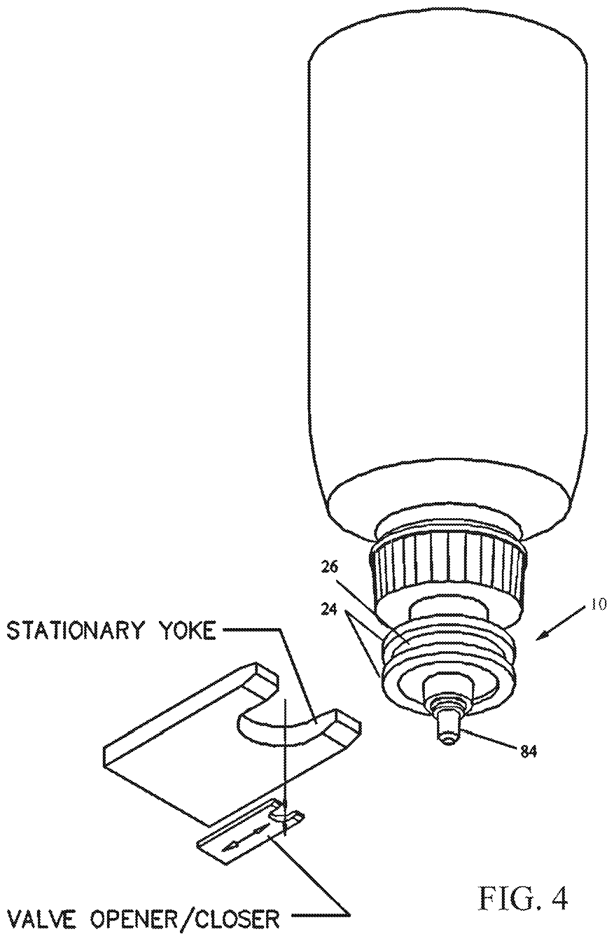

FIG. 4 is a perspective view of the push-pull adapter cap 10 of FIG. 3 illustrating how it interfaces an automated filling system. The automated filling system employs at least one anchoring yoke and at least one poppet-manipulating yoke, although one skilled in the art will recognize that counter-opposed pairs of such yokes may be used. The anchoring yoke(s) fit within the annular groove 26 of stationary portion 12 described above to thereby securely hold the medicine container by its manufacturer-supplied cap in a fixed position. The poppet-manipulating yoke fits within the annular groove 88 of poppet 84 described above to thereby insert and/or extract the poppet 84 from the stationary portion 12, thereby closing or opening the valve.

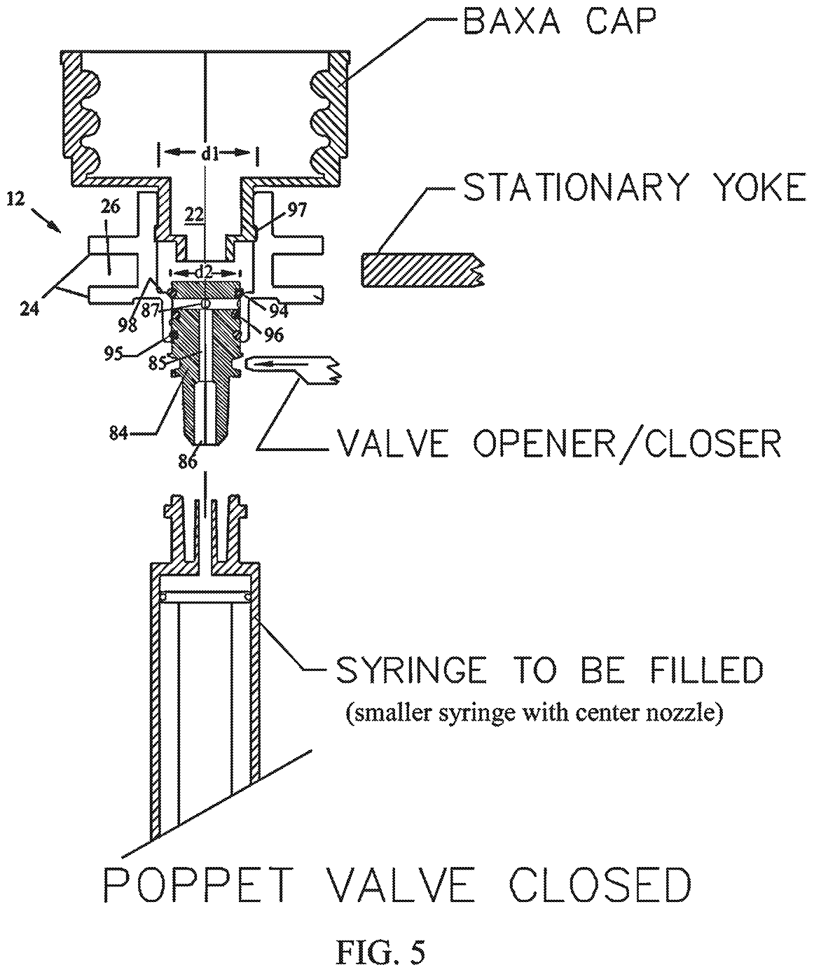

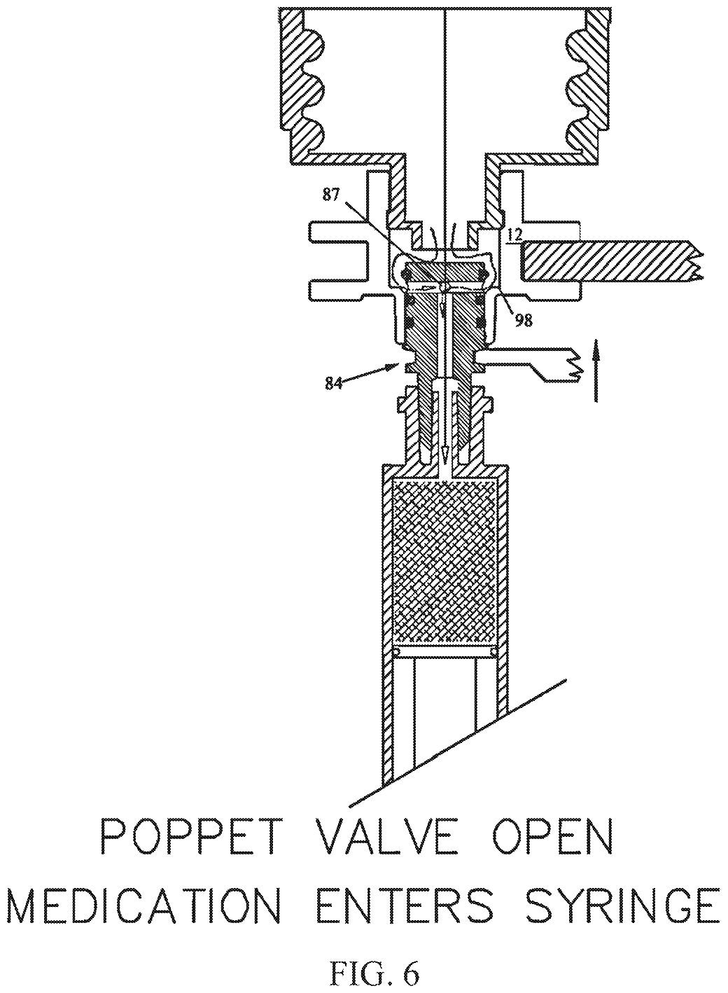

FIGS. 5-6 are sequential cross-sections of the push-pull adapter cap 10 of FIGS. 3-4 illustrating how it interfaces an enteral syringe while in the automated filling system. Note that the syringe in FIGS. 5-6 is a smaller-size enteral syringe with luer-lock enteral fitting and internal tapered slip-nozzle. In adapter cap 10 the central passage 22 continues approximately two-thirds through the stationary portion 12 at a relatively constant diameter d1, to a point approximately even with the lower flange 26. However, that constant diameter passage is interrupted at about its midpoint by a shallow annular notch 97. Notch 97 provides for snap-fit capture of the OEM cap by its nozzle, which as shown is typically provided with a distal flange for snap-fit of a nozzle cap. The central passage 22 continues at constant diameter d1 to a shoulder 98 where is constricts to a smaller diameter d2 sized to accommodate the elastomer poppet 84, as will be described further herein, which protrudes from the distal end of passage 22. Poppet 84 likewise has a central passage 85 leading from a radial inlet 87 to an axial outlet 86. Note that the tip of poppet 84 is externally-tapered yet the central passage 85 is cylindrical leading to axial outlet 86. This way, the tip of poppet 84 exactly fits the moat between the enteral syringe luer-lock enteral fitting and internal tapered slip-nozzle, preventing any accumulation of medicine and undesired leakage.

In operation, when the poppet 84 is fully extracted as seen in FIG. 5 the inlet 87 is sealed within the smaller diameter d2 portion of passage 22 and forms a fluid-tight seal. Conversely, when the poppet 84 is fully inserted as seen in FIG. 6 the inlet 87 pushes above shoulder 98 and is freed within the larger diameter d1 portion of passage 22. Medicine is free to flow through the poppet 84 as seen by the arrows of FIG. 6 for filling of a syringe (below).

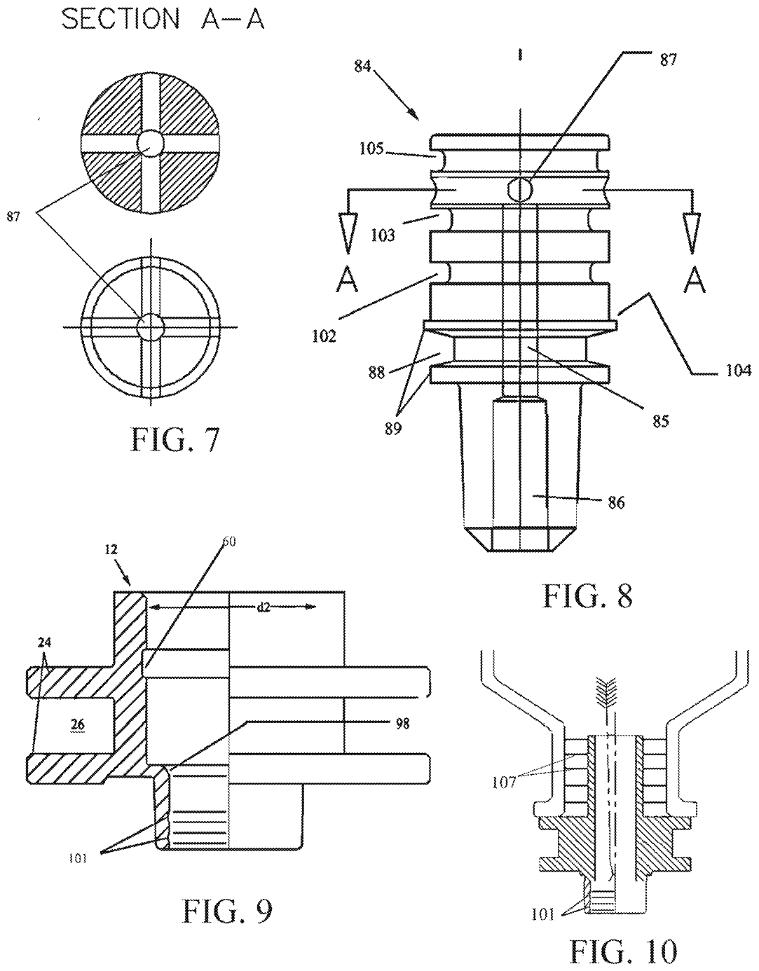

FIGS. 7 and 8 are a cross-section and side view of the poppet 84, which is an annular member formed with a plurality of grooves 101-105 spaced axially, and central passage 85 running centrally and axially and leading from a radial inlet 87 to an axial outlet 86. Radial inlet 87 may be any one or more inlet-passages extending radially into poppet 84 and in fluid communication with passage 85. Outlet 86 is enlarged and inwardly-fluted to receive and conform to the tapered nozzles of most enteral syringes. The grooves 101-105 include a lowermost groove 101 for receiving the smaller articulating yoke of the filling system which thereby inserts and/or extracts the poppet 84 from the stationary portion 12, thereby closing or opening the valve. In addition, three grooves 102, 103 and 105 are provided for three O-rings, inclusive of a groove 105 for a poppet seal 94 above the inlet 87 for sealing the poppet 84 against the shoulder 98 when fully open, and two lower grooves 103, 105 below the inlet 87 for fluid seals 95, 96 thereby preventing leakage out around poppet 84.

FIG. 9 is a side cross-section of the press-on ring 60 used for attaching the push-pull adapter cap 10 of FIGS. 3-6 to a standard manufacturer-supplied medication container cap. It is noteworthy that distal end of stationary portion 12 forms a nozzle through which the smaller diameter d2 portion of passage 22 passes, and the inner walls of this nozzle are formed with two spaced grooves forming O-ring seats 101 as shown for seating the fluid seals 95, 96 of poppet 84 when it is in its fully extracted position, and thereby ensuring a fluid-tight closure.

At the distal end of stationary portion 12 the outer diameter of the nozzle is sized to fit inside the female nozzle of a Luer lock oral syringe. The fluted outlet 86 of poppet 84 is configured to receive and conform to the tapered nozzles of most enteral syringes. This particular confirmation is well-suited for attachment to all variety of oral/enteral syringes inclusive of a Luer lock syringe with or without nozzles.

One skilled in the art will understand that other configurations may be used for attachment to other medicine containers. For example, the inner wall of the adapter cap along d2 may be defined by a simple inwardly-threaded connection for screw-insertion onto the threaded container neck. Alternately, the inner wall of the adapter cap 10 along d2 may be formed with a series of integrally formed inwardly-directed circular gripping ribs for gripping the neck of a medicine container by its threads.

FIG. 10 is a side cross-section of a modified press-in version used for attaching the push-pull adapter cap 10 inside the neck of a standard manufacturer-supplied medication container cap. Rather than fitting around the medicine container neck the ring fits inside, sealing by a plurality of resilient annular ribs 107 thereby ensuring a fluid-tight closure.

Alternatively, the ring may fit outside the medicine container neck using a plurality of inwardly-directed resilient annular ribs 107 to ensure a fluid-tight closure. In this case, as the neck of a medicine container is forced into the central void, the ribs 107 engage the threads on the outside of the neck of the bottle and flex slightly to permit the threads to pass. Once past, the ribs 107 spring back toward their original position and press against the neck to engage the threads and secure the adapter cap 10 to the container. Whether male or female, the flexure of the ribs 107 permits the adapter cap 10 to accommodate size variations in outside neck diameter and thread finish, and create a fluid-tight seal without the need for a specific thread pitch. The foregoing is set forth in more detail in co-pending application Ser. No. 13/788,849 filed Mar. 7, 2013, which is herein incorporated by reference in its entirety.

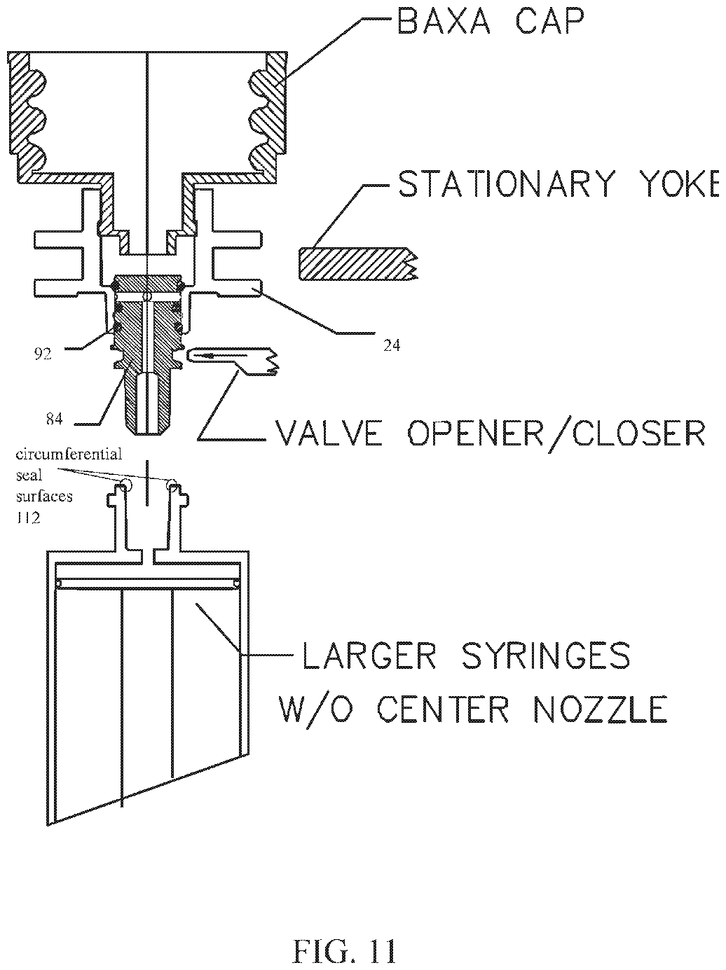

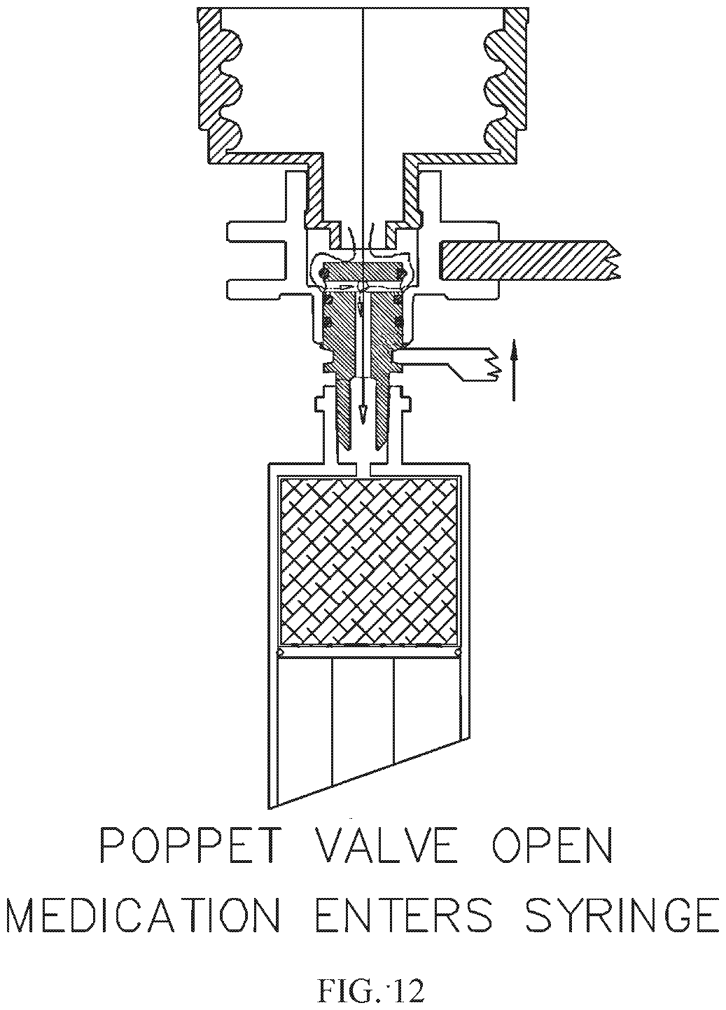

FIGS. 11-12 are sequential perspective views of the push-pull adapter cap 10 of FIGS. 3-9 illustrating how it interfaces with both the larger-size enteral syringe lacking a center nozzle, and the smaller size enteral syringe with center nozzle thereby illustrating the device's ability to adapt to both small syringes(with nozzle) and large syringes (w/o center nozzle). As above, the tip of poppet 84 is externally-tapered and exactly fits the orifice of the enteral syringe luer-lock enteral fitting preventing any accumulation of medicine in the smaller enteral syringe tip with center nozzle. Thus the tip of the poppet valve is able to be used with both the small enteral syringe with center nozzle and the larger enteral nozzle without the center nozzle.

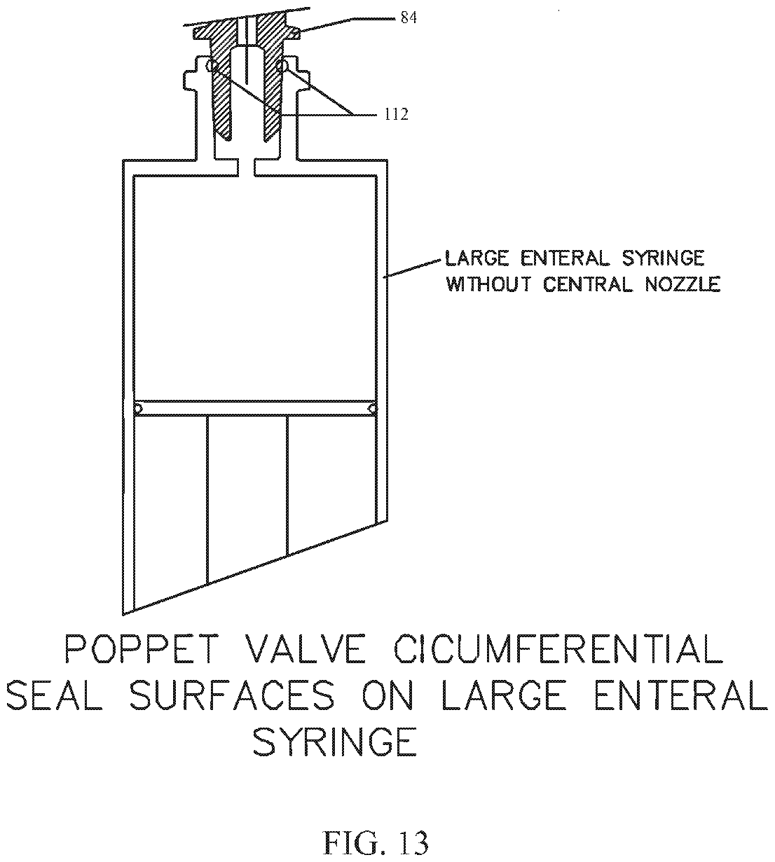

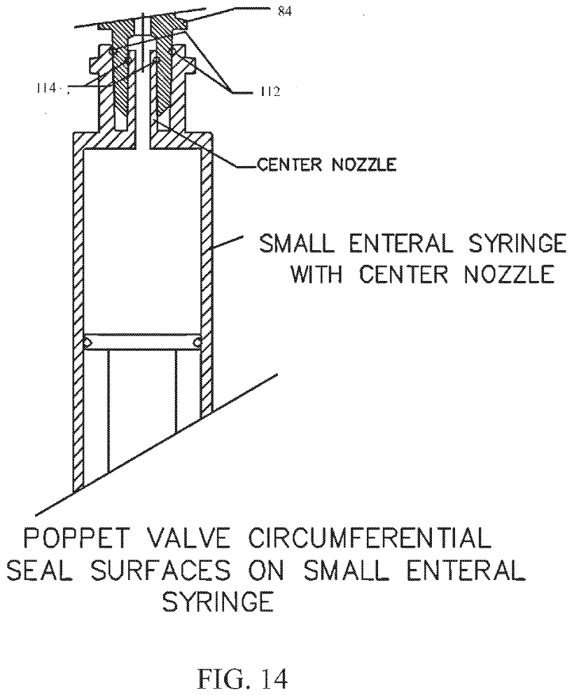

FIG. 13 is a cross-section of the poppet valve 84 of the present invention inserted into a large enteral syringe and illustrating the poppet valve circumferential seal surfaces on the large enteral syringe lacking a center nozzle. As above, the tip of poppet 84 is externally-tapered, i.e., has a tapered outer diameter that exactly mates (“taper-locks”) with the outermost internal surface of the large enteral syringe luer lock to create a first circumferential seal 112.

FIG. 14 is a cross-section of the poppet valve 84 of the present invention inserted into a small enteral syringe and illustrating the poppet valve circumferential seal surfaces on the small enteral syringe with internal center nozzle. The poppet valve 84 is configured as above for the first circumferential seal 112 plus the poppet central channel has a tapered outlet configured to mate with the outermost external surface of the enteral syringe nozzle to create a second circumferential seal 114. For the smaller enteral syringes these two seals “taper-lock” both to the outermost internal surface and to the innermost diameter of the internal nozzle of smaller enteral syringes. Despite the type of enteral syringe this configuration completely eliminates unwanted fluid buildup and inaccurate filling volumes.

In addition, molded surface features or textures may be provided on the outer surface of each cap to provide a gripping surface.

It should now be apparent that the above design interfaces with and enables opening and closing the flow of medication to enteral syringes with Luer locks and/or internal slip-nozzles while the medication container is inverted. As such, the time to load and unload, or upright and invert, the medication container between syringe fillings is eliminated. In addition, the medication container can also be shaken in an inverted position before, during or after a syringe filling operation, when the medication so requires.

In addition, a “smart” manufacturer-supplied medication container is herein disclosed for bulk medicine that bears OEM information relevant to the filling process. This way, hospital pharmacists seeking to fill oral and enteral syringes with medication on-demand at hospital pharmacies from bulk medication containers supplied by OEM manufacturers have the OEM information at their disposal when filling the syringes, saving considerable time, effort and cost.

The OEM information relevant to the filling process is preferably provided on the base of the bulk medicine container, by a label or other indicia attached thereto. One skilled in the art will understand that the label/indicia may further comprise a 2D barcode to enable easy tracking of the medication container bearing that cap, or may be an RFID tag or other label. Each barcode (or possibly RFID tag or other label) preferably references the following information:

Batch number

Expiry date

Storage instructions

Product name

Strength

Name of the active ingredient(s)

Dose form

Warning statements

NDC number (National Drug Code)

Does product need to be shaken before use? If so, how often?

Does product need to be refrigerated before use? If so, temp?

Does product need to be protected from light?

Volume of original bulk medication container?

The label/indicia containing the above OEM information is most preferably attached to the bulk medication container when it is filled by the OEM manufacturer. This way, hospital pharmacists seeking to fill oral and enteral syringes with medication on-demand at hospital pharmacies from bulk medication containers supplied by OEM manufacturers have the relevant OEM information at their disposal and can scan it or enter it into the filling system when filling syringes. This way, the OEM data is already in the filling system. This accomplishes the following: 1) avoids the need to log in the medication container at the Hospital Pharmacy and input information into the database; 2) avoids the need to replace the standard medication cap with an adapter cap, inasmuch as the adapted cap can be placed on the container by the OEM manufacturer at the outset, saving labor and avoiding transcription errors by a Pharmacy Technician; 3) saves the cost of the standard medication cap (assuming the aforesaid adapter cap is installed by the OEM manufacturer at the time that it is filled; 4) reduces the possibility of the Pharmacy Technician contaminating the contents of the bulk medication container while replacing the standard medication cap with the adapter cap; and 5) reduces the risk of leakage if the OEM manufacturer installs the adapter cap and then a “leak-proof” seal overtop. None of this would not be possible if the adapter caps are installed by the Pharmacy Technician.

Having now fully set forth the preferred embodiment and certain modifications of the concept underlying the present invention, various other embodiments as well as certain variations and modifications of the embodiments herein shown and described will obviously occur to those skilled in the art upon becoming familiar with said underlying concept. It is to be understood, therefore, that the invention may be practiced otherwise than as specifically set forth in the appended claims.

We claim:

1. A method of filling of a syringe with medication, comprising the steps of:
loading a container of medicine into a loading station of a filling system, said medication container comprising a closure having a push-pull nozzle;
inverting the medication container;
loading a syringe to fill with medicine from said container into said loading station of said filling system;
inserting said syringe into said push-pull nozzle of the medication container;
opening said push-pull nozzle of said the medication container closure;
withdrawing a plunger of said syringe to thereby fill said syringe with medicine from said medicine container;
closing said push-pull nozzle; and
withdrawing the syringe from the medication container.

2. The method of claim 1, wherein said syringe is any one from among the group of an enteral syringe, an oral syringe.

3. The method of claim 1, wherein said step of opening said push-pull nozzle comprises slidably withdrawing said push-pull nozzle.

4. The method of claim 1, further comprising a step of installing said closure onto said medicine container by screw-attachment.

5. The method of claim 1, further comprising a step of installing said closure onto said medicine container by press-fit insertion.

6. The method of claim 1, wherein said step of inverting the medication container comprises gripping said adapter cap by a first flange protruding laterally from and encircling said annular member.

7. The method of claim 3, wherein said step of opening said push-pull nozzle comprises gripping a flange protruding laterally from and encircling said annular member.

8. The method of claim 1, wherein said step of attaching a syringe comprises inserting said syringe into an inwardly tapered radial inlet of said annular member.

9. A method for preparing medication containers for automated filling of syringes, comprising the steps of,
receiving a plurality of medication containers,
filling said plurality of medication containers with medicine from a singular batch having a known batch lot code;
entering said batch lot code into a database;
installing a cap on each of said plurality of medication containers, said caps all comprising a syringe filling interface for automated filling of a syringe;
assigning a unique identifier to each of said plurality of medication containers;
entering said unique identifier into said database in association with said batch lot code, printing a plurality of labels all indicating said known batch lot code and a unique identifier; and
placing the plurality of labels on the plurality of medication containers.

10. The method of claim 9, further comprising a step of scanning each of said plurality of labels and verifying that the contents are correct.

11. A method for filling syringes with medication from a medication container, comprising the steps of:
attaching an adapter cap to a medication container, said adapter cap having a stationary portion comprising an annular base and neck having a central channel, and a poppet slidably inserted into the neck;
inverting the medication container;
inserting said syringe into said adapter cap;
opening said poppet; and
withdrawing a plunger of said syringe to thereby fill said syringe with medicine from said medicine container.

12. The method of claim 11, further comprising a step of closing said push-pull nozzle.

13. The method of claim 12, further comprising a step of withdrawing the syringe from the medication container.

14. The method of claim 11, wherein said syringe is any one from among the group of an enteral syringe, and an oral syringe. =.

15. The method of claim 11, wherein said step of inverting the medication container comprises gripping said adapter cap by a flange.

16. The method of claim 14, wherein said step of opening said poppet comprises gripping said poppet by a flange.

17. The method of claim 10, wherein said step of attaching a syringe comprises inserting said syringe into an inwardly tapered radial inlet of said adapter cap.

18. A method of filling of a syringe with medication from a medicine container comprising the steps of:
providing a medicine container having a closure with a push-pull valve;
inverting the medication container;
attaching a syringe for filling the syringe with medication from said medicine container;
opening said closure push-pull valve;
withdrawing a plunger of said syringe to thereby fill said syringe with medicine from said medicine container;
closing said closure push-pull valve; and
withdrawing the syringe from the medication container.

19. The method of claim 18, wherein said step of opening said push-pull valve comprises slidably withdrawing said push-pull valve.

20. The method of claim 18, wherein said step of providing a medicine container comprises installing an adapter cap on said medicine container.

21. The method of claim 18, wherein said step of opening said push-pull valve comprises gripping said push-pull valve by at least one projecting flange.

22. The method of claim 18, wherein said step of attaching a syringe comprises inserting said syringe into an inwardly tapered radial inlet of said closure.

23. The method of claim 18, wherein said step of inverting the medication container comprises gripping said closure by a first projecting flange protruding laterally from and encircling said closure.

24. The method of claim 23, wherein said step of opening said poppet comprises gripping said poppet by a second projecting flange protruding laterally from and encircling said poppet.

25. A method of filling a syringe with medication, comprising loading a container of medicine into a filling system, loading a syringe to fill with medicine from said container into said filling system, inserting said syringe into a closure of said medication container, opening a valve of said medicine container, withdrawing a plunger of said syringe to thereby fill said syringe with medicine from said medicine container, and withdrawing the syringe from the medication container.

26. The method of claim 25, wherein said syringe is any one from among the group of an enteral syringe, an oral syringe.

27. The method of claim 25, wherein said valve is a push-pull valve.

28. The method of claim 27, wherein said step of opening a valve of said medicine container comprises gripping said valve by flange protruding laterally therefrom.

29. The method of claim 25, wherein said step of inserting said syringe comprises inserting said syringe into an inwardly tapered radial inlet of said closure.

30. A method for manufacturing medicine containers for use in automated filling of syringes with medicines from said containers, said syringes being of various sizes and types all having a barrel, an annular flange encircling said barrel, a plunger slideably engaged in said barrel, and a flange at distal end of said plunger, the method comprising the steps of:
assigning a batch code to a batch of prescription medicine;
storing said batch code in a database;
filling a medicine container with prescription medicine from said batch;
installing a cap on said filled medicine container, said cap having a leak-proof valve for insertion of a syringe;
assigning a unique identification code to said filled and capped medicine container;
storing said unique identification code in a database in association with said batch code; and
attaching an indicia to said medicine container identifying said batch code and unique identification code.

31. The method for manufacturing medicine containers according to claim 30, wherein said indicia comprises a 2D barcode.

32. The method for manufacturing medicine containers according to claim 30, wherein said indicia comprises an RFID tag.

33. The method for manufacturing medicine containers according to claim 30, wherein said valve comprises a push-pull valve.

34. The method for manufacturing medicine containers according to claim 30, wherein said valve comprises a duck bill valve.

35. The method for manufacturing medicine containers according to claim 30, further comprising a step of storing supplemental information in said database comprising any one or more of a batch expiry date, storage instructions, National Drug Code, whether to shake before use, whether to refrigerate, whether to protect from light, and volume of medication container.
